(19) United States
(12) Patent Application Publication
(10) Pub. No.: US 2010/0260689 A1
(43) Pub. Date: Oct. 14, 2010

(54) REDUCING SHAVING FREQUENCY

(75) Inventors: Katrin Wilke, Sauensiek (DE); Bjoerna Windisch, Hamburg (DE); Peter Maurer, Neumuenster (DE); Claudia Mueller, Tangstedt (DE); Norman Lipinski, Hamburg (DE); Michael Urban, Hamburg (DE); Nicole Muehlen, Trittau (DE); Sabine Howe, Hamburg (DE); Annika Mannes, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 12/740,504

(22) PCT Filed: Oct. 27, 2008
(86) PCT No.: PCT/EP08/09066

§ 371 Date: Jul. 1, 2010

(30) Foreign Application Priority Data
Nov. 2, 2007 (DE) 10 2007 052 865.7

(51) Int. Cl. A61K 8/02; A61K 8/92; A61Q 9/02

(52) U.S. Cl. 424/47; 424/73; 424/68; 132/200

(57) ABSTRACT

The invention describes the use of acidic aluminum compounds that in cosmetic preparations result in hair softening and a reduction of the shaving frequency.

The invention describes the use of acidic aluminum compounds, which in cosmetic preparations cause the hair to soften, reduce the cutting force and lead to a reduction in the shaving frequency.

Personal hygiene, and in particular skin care, increasingly includes shaving in addition to cleansing the skin and skin care.

In addition to a man's beard growth, men as well as women regularly remove hair on the legs and above all underarm hair.

Various means, such as epilation means, and preparations are known for this purpose.

The key factor with all of the known means is to increase the smoothness of the skin and to keep the skin smooth for as long as possible. It is desirable thereby to minimize shaving frequency, i.e., the frequency of hair removal.

This is mainly because the skin is frequently irritated by shaving or hair removal. Daily hair removal is sometimes rendered impossible as a result or is associated with marked skin irritation.

Experience has shown that frequent shaving, in particular in the underarm region, leads to skin soreness and skin irritation. The prior art attempted to eliminate this drawback by means of hair growth inhibitors.

For example, papaya is described as a hair growth inhibitor in protective right WO 2006125582.

WO 200730139 describes cosmetic preparations containing behenoxy dimethicone. These preparations are intended to have an improved skin compatibility and to help to reduce the visibility of hair and beard stubble.

EP 1588690 describes mixtures and methods for delaying or preventing undesirable hair growth, wherein the mixture comprises malic acid, an electrolyte and a water-based agent that is acceptable in cosmetics and includes a film-forming agent.

U.S. Pat. No. 7,238,343 B2 describes deodorants and antiperspirants that contain natural oils, in particular sunflower oil, and glycerol in a ratio of 4:1 to 1:4. The use of preparations of this type is intended to improve the regeneration of the skin after hair removal.

WO 2006125582 A1 describes antiperspirant emulsions with hair growth inhibitors.

Shaving aids, foams and gels are based on alkaline preparations, since it is known that soap-based and thus alkaline preparations cause hairs to swell, and thus enable a more thorough shave.

The above-referenced shaving preparations are sometimes added to shaving aids. These are particulate compounds or water-soluble polymers, such as PEG-14M, PEG-7M, PEG-90M (=PEG 90000). PEG 45M (polymers of ethylene oxide with average degrees of polymerization as indicated multiplied by a factor of 1000), acrylates, PTFE (polytetrafluoroethylene) or cellulose derivatives that are suitable for improving sliding properties.

Known cosmetic shaving preparations often have a substance that improves sliding properties from the group of ethoxylated polywaxes, such as, e.g., PEG-6000 as an ingredient. The use of PEG compounds in cosmetic preparations is very controversial, however. One reason for concern is the possibility of promoting the penetration of potentially irritative or allergenic constituents of the cosmetics.

The PEG compounds are a large group of very different substances, which are not uniform in terms of their penetration promoting properties.

The object of the present invention is therefore to provide cosmetic shaving preparations, which prepare the skin in the armpits for shaving and which improve the sliding and cutting properties during shaving.

It is desirable thereby to omit penetration promoting constituents or to limit their content.

In addition, the shaving preparations from the prior art are often not tolerated well by the skin.

Another drawback is that a variety of compositions are available. Thus, deodorant or antiperspirant products which are to be used in parallel or consecutively in addition to the shaving aids.

In addition to the reduction in hair growth, above all skin tolerance is a crucial attribute of an efficient deodorant/antiperspirant accepted by customers.

The invention now takes a different approach in which the shaving frequency and skin tolerance are the focus, not the reduction of hair growth.

Surprisingly, it has been shown that acidic aluminum compounds in cosmetic preparations help to reduce shaving frequency.

According to the invention, acidic aluminum compounds are used to produce topically applicable preparations for reducing shaving frequency.

Furthermore, acidic aluminum compounds are used to produce topically applicable preparations for softening human hair, in particular armpit hair, according to the invention.

The use of acidic aluminum compounds to produce preparations leads to a reduction in the shaving cutting force after the topical application of the preparation.

The sliding force with which the blade is moved over the surface of the skin also has particular importance for a cosmetic shaving preparation. It should be as low as possible.

Furthermore, the preparation should be absorbed well onto the skin and less well onto the razor, so that the razor does not clog or become blunt more quickly. The shaving preparation is also designed to stabilize the hair, i.e., to straighten it in a way so that it can be cut off deeper. Just after this fresh cut is when the sore, mechanically irritated skin is to be smoothed and soothed.

The shaving preparation according to the invention solves this host of problems.

Comparative tests show that acidic aluminum compounds, such as, e.g., aluminum chlorohydrates, lead to a reduction of the cutting force of the hair during shaving.

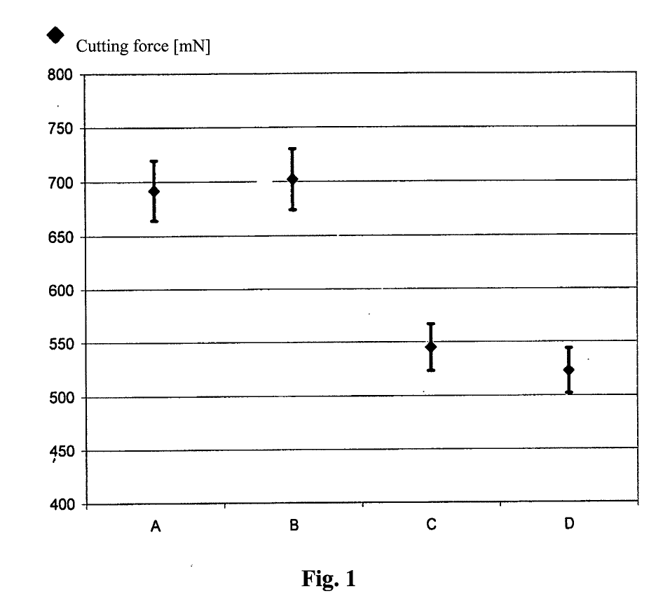

FIG. 1 shows the cutting force during the shaving of human hair after 24 hours of exposure time to the products A through D.

The sliding of the razor blade can be physically divided into dry friction and fluid friction. Coulomb dry friction is thereby influenced by the friction coefficient of the substrate, the viscous friction between or the sliding of the fluid films is thereby determined largely by the dynamic viscosity.

The cutting force, i.e., the force that is necessary in order to cut off a hair, is also of particular importance with shaving.

With respect to materials engineering, shaving involves a separation process. Cutting beard stubble is best described by the principle of wedge severing. The cutting force FB_{SB} can thereby be described with the aid of the following formula:

F_{S}=l_{S}·s·k_{Ss}

F_{S}=cutting force k_{S}≈0.8·Rₘ
l_{S}=cutting line length Rₘ=tensile strength
s=cut thickness/diameter
k_{S}=shear strength

The formula given above thereby applies to a 90° cut with a fixed substrate.

The measurements are thereby carried out on a universal test apparatus with cutting force adapter with four friction bearings and recorded by means of TestXpert software.

Five hair fibers were cut for the measurement.

Due to the influence of the cross section of the hair fiber, a preliminary grouping of the hair fibers to be cut is indispensable, which ensures that the products to be compared were carried out on hair fibers with the same cross section so that relative results are obtained.

Measurement Set-Up

Five hair fibers fixed on one side are cut with the aid of a commercially available blade (single-blade razor with replaceable blade) at a constant speed. The blade is thereby clamped in an adapter specially produced for this purpose, which ensures a guidance of the blade with the aid of four friction bearings. The determination of the cutting force is carried out by maximum force detection. At the same time, the friction force of the friction bearings is recorded during each measurement. Since the cutting forces are directly dependent on the cross-sectional surface, it must be ensured before each measurement that the distribution of the hair fiber cross sections between different groups is comparable.

Measurement parameters: Measurement speed: 100 mm/min
Cutting angle: 90°
Blade grind: approx. 12°

A cosmetic preparation, a shaving preparation, can thereby influence the sliding and cutting forces.

The tested products A-D (see FIG. 1) comprise an artificial sweat solution composed of

Starting eluent: 1 liter (distilled water)
500 mg glycine
1300 mg lactic acid (90% aq)
100 mg urea
1800 mg NaCl
480 mg KCl
1000 mg ammonium chloride
   pH value: approx. 3.0

based on the mean values of sweat analyses of male and female test subjects—as well as:

A: No further constituents
B: Citric acid (aqueous solution, pH=4.45)
C: Aluminum chlorohydrate (ACH, 10% in aqueous solution, pH=4.45)
D: Aluminum chlorohydrate (ACH, 10% in aqueous solution, pH=4.45) and avocado oil (0.1% by weight)

The preparations C and D represent preferred preparations according to the invention.

The fact that the cutting force reduction by ACH is not based on an influence by the pH value, is proven by the comparison preparation B, to which citric acid was added in order to obtain a similar pH value as the preparations containing ACH.

The finding was surprising that acidic aluminum compounds lead to a softening of the hair, which in turn leads to the measured cutting force reduction.

It is known that alkaline preparations soften the hair and thus render possible a full shave. Therefore, until now only alkaline preparations, such as the known shaving soaps, etc., have been considered suitable for a thorough shave.

According to the invention, however, surprisingly acidic aluminum compounds are now suitable in cosmetic preparations as shaving aids.

Acidic means according to the invention a pH value in aqueous medium of 7 and less.

Acidic aluminum compounds, in particular aluminum chlorohydrates, are thus to be used to produce topically applicable preparations for softening the human hair, in particular underarm hair.

Likewise, the use of acidic aluminum compounds, in particular aluminum chlorohydrates, is predestined to be used to produce preparations to reduce the shaving cutting force after topical application of the preparation.

The basic principle is that the preparations according to the invention comprising acidic aluminum salts, in particular aluminum chlorohydrates, reduce the cutting force during shaving.

If additionally skin care or skin softening additives, such as for example, oils, are added to the preparation according to the invention, a further improved shave or reduction in the cutting force during shaving (see D, FIG. 1) is shown.

Aluminum chlorohydrates are known antiperspirants. However, to use them now to apply the preparations containing them to the areas of skin to be shaved in order to improve a shave was surprising and not foreseeable.

Through a more thorough shave, the frequency with which a new shave must take place is thus reduced.

The addition of oils, in particular avocado oil, to cosmetic preparations is known. According to the invention, the addition, however, leads to a further improved thoroughness, as well as a reduction in the cutting force.

Avocado oil is considered to be softening and smoothing. Avocado oil and/or the lipids octyldodecanol, caprylyl carbonate and/or paraffinum liquidum are particularly advantageously contained in the preparations according to the invention as oil components.

Advantageously, additional substances for reducing hair growth can be added to the preparation. Preferred substances are to be selected from the group papaya extract, carica papaya, celandine extract and/or chelidonium majus extract.

The preparations according to the invention are preferably emulsion-based preparations. The preparations according to the invention can also be provided as a lotion, cream, spray, aerosol, as a roll-on or stick or foam preparations.

The preparations are preferably formulated and provided as an aerosol, roll-on or stick.

One skilled in the art can select the accompanying substances necessary for this purpose, such as propellant gas for aerosols, from the known agents.

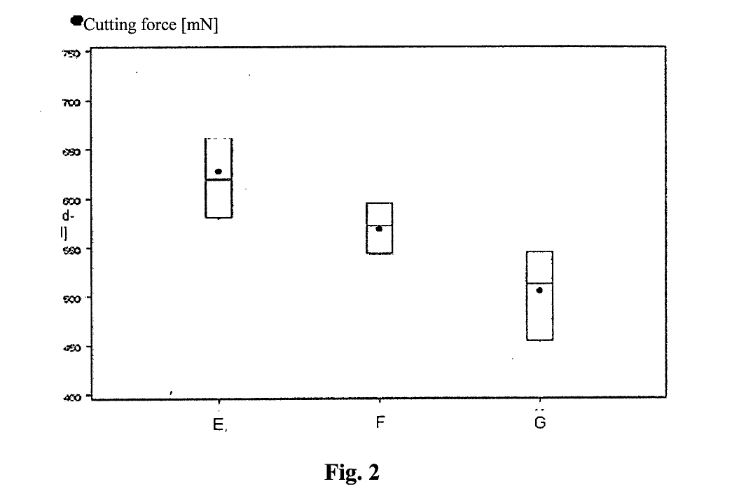

In a further comparative test, the cutting force on human hair was measured after 24 hours of exposure time of three preparations (FIG. 2).

The tested preparations E-G (see FIG. 2) comprise a pure artificial sweat solution (like preparation A).

E: No further constituents
F and G: Additionally an emulsion-based preparation as a roll-on with
F: as macroemulsion

| | % (w/w) |
|---|---|
| Aqua | ad 100 |
| Aqua + trisodium EDTA | 1.5 |
| Persea gratissima oil | 0.25 |
| Steareth-21 | 1.5 |
| Steareth-2 | 2.5 |
| PPG-15 stearyl ether | 3 |
| Aluminum chlorohydrate (50% aq) | 20 |
| Perfume | 1 |

G: as microemulsion with paraffin oil

| | % (w/w) |
|---|---|
| Aqua | ad 100 |
| Octyldodecanol | 3 |
| Paraffinum liquidum | 3 |
| Persea gratissima oil | 0.1 |
| Glyceryl isostearate | 2 |
| Glycerin | 2.6 |
| Aluminum chlorohydrate (50% aq) | 20 |
| PEG-150 distearate | 0.7 |
| Isoceteth-20 | 4 |
| Perfume | 1 |

The preparations F and G represent preferred preparations according to the invention and show the advantageous use of emulsion-based preparations with acidic aluminum compounds to soften hair, to reduce cutting force and to reduce shaving frequency.

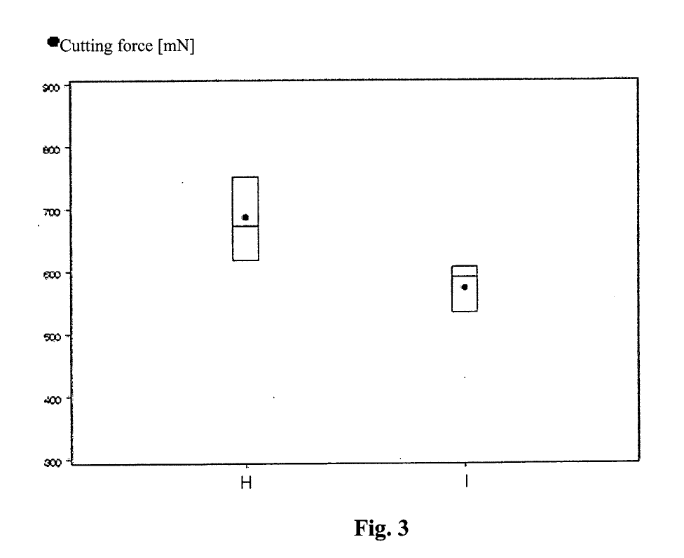

In a further comparative test, the cutting force on human hair was measured after 24 hours of exposure time of three preparations (FIG. 3).

The tested preparations H-I (see FIG. 3) comprise

H: a pure artificial sweat solution (like A)
I: additionally an emulsion-based preparation as a roll-on with
I: as a microemulsion with dicaprylyl carbonate

| INCI | % (w/w) |
|---|---|
| Aqua | ad 100 |
| Octyldodecanol | 3 |
| Persea gratissima oil | 0.1 |
| Glyceryl isostearate | 2 |
| Glycerin | 2.6 |
| Aluminum chlorohydrate | 20 |
| PEG-150 distearate | 0.7 |
| Isoceteth-20 | 4 |
| Dicaprylyl carbonate | 3 |
| Perfume | 1 |

The preparation I represents a preferred preparation according to the invention.

Preferred preparations are emulsion-based preparations, in particular formulated as a macroemulsion or a microemulsion, advantageously with additional paraffin oil and/or dicaprylyl carbonate.

The preparations according to the invention are advantageously to be used as a roll-on.

In addition, use as a W/O spray is also preferred according to the invention.

The preparations are preferably alcohol-free, surfactant-free and/or soap-free for reasons of skin compatibility.

Surfactant-free does not thereby mean that no emulsifiers are included.

Shaving aids are dry razors or wet razors, metallic blades or knives.

The compositions according to the invention may also optionally contain additional additives as are customary in cosmetics, for example, perfume, thickeners, deodorants, antimicrobial agents, refatting agents, complexing and sequestering agents, pearlescent agents, plant extracts, vitamins, active ingredients, preservatives, bactericides, dyes, pigments that have a coloring effect, thickeners, moisturizers and/or humectants, fats, oils, waxes, unless excluded, or other customary constituents of a cosmetic or dermatological formulation, such as alcohols, polyols, polymers, foam stabilizers, electrolytes, organic solvents or silicone derivatives.

Moisturizers can also advantageously be used as anti-wrinkle ingredients to protect against skin changes, such as occur, e.g., in the aging of the skin.

Constituents that represent a penetration promotion for potentially irritative or allergenic constituents, such as, for example, ethoxylated polywaxes, such as, e.g., PEG-6000, can be omitted according to the invention.

The preparations according to the invention comprising in particular aluminum compounds with antiperspirant effect, such as aluminum chlorohydrates, in addition to the described reduction in shaving frequency lead to a reduction in the cutting force and thus to a shave that is gentler to the skin and also to a reduction in sweat formation.

Thus several cosmetic tasks are performed with a single preparation. In addition to on average 24-hour antiperspirant effectiveness, in parallel the skin is prepared for a more thorough shave and in addition the skin is treated, in particular when oils, such as avocado oil, octyldodecanol, caprylyl carbonate and/or paraffinum liquid are contained.

The area of application for the preparations according to the invention is preferably the armpit region, the legs as well as a person's genital and chest area.

The following examples show preferred embodiments of the formulations according to the invention. Unless stated otherwise, the numerical values relate to weight percentages, based on the total weight of the formulations.

| | Example 1 |
|---|---|
| Aqua | 63.6 |
| Octyldodecanol | 3 |
| Persea gratissima oil | 0.1 |
| Glyceryl isostearate | 2 |
| Glycerin | 2.6 |
| Aluminum chlorohydrate | 20 |
| PEG-150 distearate | 0.7 |
| Isoceteth-20 | 4 |
| Dicaprylyl carbonate | 3 |
| Perfume | 1 |

### EXAMPLE 2

### Roll-on (Emulsion, Milky White)

| | % (w/w) |
|---|---|
| Aqua | ad 100 |
| Aqua + trisodium EDTA | 1.5 |
| Persea gratissima oil | 0.25 |
| Steareth-21 | 1.5 |
| Steareth-2 | 2.5 |
| PPG-15 Stearyl ether | 3 |
| Aluminum chlorohydrate (50% aq) | 20 |
| Perfume | 1 |

### EXAMPLE 3

### Roll-on (Emulsion, Translucent)

| | % (w/w) |
|---|---|
| Aqua | ad 100 |
| Octyldodecanol | 3 |
| Paraffinum liquidum | 3 |
| Persea gratissima oil | 0.1 |
| Glyceryl isostearate | 2 |
| Glycerin | 2.6 |
| Aluminum chlorohydrate (50% aq) | 20 |
| PEG-150 distearate | 0.7 |
| Isoceteth-20 | 4 |
| Perfume | 1 |

### EXAMPLE 4

### Roll-on (Emulsion, Translucent)

| | % (w/w) |
|---|---|
| Aqua | ad 100 |
| Octyldodecanol | 3 |
| Persea gratissima oil | 0.1 |
| Glyceryl isostearate | 2 |
| Glycerin | 2.6 |
| Aluminum chlorohydrate (50% aq) | 20 |
| PEG-150 distearate | 0.7 |
| Isoceteth-20 | 4 |
| Dicaprylyl carbonate | 3 |
| Perfume | 1 |

### EXAMPLE 5

### W/O Spray (Aerosol)

| | % (w/w) in WL |
|---|---|
| Aqua | ad 100 |
| Preservatives | qs |
| Persea gratissima oil | 0.5 |
| C12-15 alkyl benzoates | 5 |
| Cetyl PEG/PPG-10/1 dimethicone | 1.5 |
| Aluminum chlorohydrate (50% aq) | 15-30 |
| Polysorbate 65 | 1 |
| Dicaprylyl ether | 4 |
| Polyglyceryl-2 dipolyhydroxy stearate | 1.5 |
| Butyloctanoic acid | 0.25 |
| Cyclomethicone | 11.75 |
| Dicaprylyl carbonate | 5 |
| Perfume | qs |

Filling as aerosol with propellant gas (propane/butane).

### EXAMPLE 6

### Antiperspirant Stick

| | % (w/w) |
|---|---|
| Octyldodecanol | 0.1 |
| Glyceryl stearate SE | 0.6 |
| Persea gratissima oil | 0.5 |
| Hydrogenated castor oil | 1.5 |
| Talc | 4 |
| PPG-14 butyl ether | 15 |
| Cyclomethicone | ad 100 |
| Aluminum zirconium tetrachlorohydrex | 10-20 |
| GLY | |
| Stearyl alcohol | 20 |
| Perfume | qs |

1.-9. (canceled)

10. A method of shaving human skin, wherein the method comprises (a) topically applying to skin to be shaved a cosmetic composition which comprises at least one acidic aluminum compound and (b) shaving the skin.

11. The method of claim 10, wherein the cosmetic composition at least one of results in a reduction of a required shaving frequency, a softening of hair to be shaved, and a reduction in a shaving cutting force.

12. The method of claim 10, wherein armpit hair is shaved.

13. The method of claim 10, wherein the composition further comprises one or more lipids.

14. The method of claim 13, wherein the one or more lipids comprise at least one of avocado oil, octyldodecanol, caprylyl carbonate, and paraffinum liquidum.

15. The method of claim 13, wherein the one or more lipids comprise avocado oil.

16. The method of claim 10, wherein the composition is at least one of alcohol-free, surfactant-free, and soap-free.

17. The method of claim 10, wherein the composition comprises an emulsion.

18. The method of claim 10, wherein the at least one acidic aluminum compound comprises one or more aluminum compounds having an antiperspirant effect.

19. The method of claim 18, wherein the at least one acidic aluminum compound comprises at least one aluminum chorohydrate.

20. The method of claim 10, wherein the composition is used as at least one of an aerosol, a roll-on, and a stick.

21. A topical cosmetic composition, wherein the composition is suitable for preparing skin for shaving, comprises at least one acidic aluminum compound and at least one lipid and, upon topical application to skin to be shaved is capable of at least one of reducing a required shaving frequency, softening of hair to be shaved, and reducing a shaving cutting force.

22. The composition of claim 21, wherein the one or more lipids comprise at least one of avocado oil, octyldodecanol, caprylyl carbonate, and paraffinum liquidum.

23. The composition of claim 21, wherein the one or more lipids comprise avocado oil.

24. The composition of claim 21, wherein the composition is at least one of alcohol-free, surfactant-free, and soap-free.

25. The composition of claim 21, wherein the composition comprises an emulsion.

26. The composition of claim 21, wherein the at least one acidic aluminum compound comprises one or more aluminum compounds having an antiperspirant effect.

27. The composition of claim 26, wherein the at least one acidic aluminum compound comprises at least one aluminum chorohydrate.

28. The composition of claim 21, wherein the composition is present as at least one of an aerosol, a roll-on, and a stick.

29. A topical cosmetic composition, wherein the composition is suitable for preparing skin for shaving, comprises at least one aluminum chlorohydrate and one or more lipids which comprise at least one of avocado oil, octyldodecanol, caprylyl carbonate, and paraffinum liquidum, and is at least one of alcohol-free, surfactant-free, and soap-free.
